(19) United States
(12) Patent Application Publication
(10) Pub. No.: US 2026/0207694 A1
(43) Pub. Date: Jul. 23, 2026

(54) PULICARIA INCISA INFUSION FOR REDUCING NEURONAL CELL DEATH AND TREATING AGERELATED NEURONAL PATHOLOGIES

(71) Applicant: THE STATE OF ISRAEL, MINISTRY OF AGRICULTURE & RURAL DEVELOPMENT, AGRICULTURAL RESEARCH ORGANIZATION, Rishon Lezion (IL)

(72) Inventor: Anat ELMANN, Rehovot (IL)

(21) Appl. No.: 19/139,733

(22) PCT Filed: Dec. 14, 2023
(86) PCT No.: PCT/IL2023/051269

§ 371 Date: Jun. 16, 2025

Related U.S. Application Data
(60) Provisional application No. 63/433,079, filed on Dec. 16, 2022.

(51) Int. Cl. A61K 36/28; A61K 9/00; A61P 25/22; A61P 25/28

(52) U.S. Cl. CPC A61K 36/28; A61K 9/0053; A61P 25/22; A61P 25/28

(57) ABSTRACT

The invention concerns pharmaceutical and nutritional compositions comprising an infusion of the plant Pulicaria incisa (Pi) for preventing, attenuating, or ameliorating the symptoms of neuronal cell death. Particularly neuronal cell death associated with an age-related neuronal pathology, including normal ageing of the brain.

### TECHNOLOGICAL FIELD

The present disclosure generally relates to a novel treatment for brain damage.

### BACKGROUND ART

References considered to be relevant as background to the presently disclosed subject matter are listed below:
1. Collin, F., Chemical Basis of Reactive Oxygen Species Reactivity and Involvement in Neurodegenerative Diseases. Int J Mol Sci, 2019. 20(10).
2. Salim, S., Oxidative Stress and the Central Nervous System. J Pharmacol Exp Ther, 2017. 360(1): p. 201-205.
3. Harman, D., Aging: a theory based on free radical and radiation chemistry. J Gerontol, 1956. 11(3): p. 298-300.
4. Franzoni, F., et al., Oxidative Stress and Cognitive Decline: The Neuroprotective Role of Natural Antioxidants. Frontiers in Neuroscience, 2021. 15.
5. Cui, X., Q. Lin, and Y. Liang, Plant-Derived Antioxidants Protect the Nervous System from Aging by Inhibiting Oxidative Stress. Frontiers in Aging Neuroscience, 2020. 12.
6. Sies, H., Hydrogen peroxide as a central redox signaling molecule in physiological oxidative stress: Oxidative eustress. Redox Biol, 2017. 11: p. 613-619.
7. Marinho, H. S., et al., Hydrogen peroxide sensing, signaling and regulation of transcription factors. Redox Biology, 2014. 2: p. 535-562.
8. Elmann, A., et al., Pulicaria incisa infusion attenuates inflammatory responses of brain microglial cells. Journal of Functional Foods, 2016. 25: p. 110-122.
9. Elmann, A., et al., Antioxidant and astroprotective effects of a Pulicaria incisa infusion. Oxid Med Cell Longev, 2012. 2012: p. 157598.
10. Mansour, R. M. A., et al., The flavonoids of Pulicaria incisa. Fitoterapia, 1990. 61: p. 186-187.
11. Hyslop, P. A., et al., Measurement of striatal H2O2 by microdialysis following global forebrain ischemia and reperfusion in the rat: correlation with the cytotoxic potential of H2O2 in vitro. Brain Res, 1995. 671(2): p. 181-6.

Acknowledgement of the above references herein is not to be inferred as meaning that these are in any way relevant to the patentability of the presently disclosed subject matter.

### BACKGROUND

Increased lifespans in the Western world have led to a rise in the frequencies of neurodegenerative diseases resulting from neuronal loss. Despite their increased prevalence, there are currently no effective preventive or therapeutic modalities for these deficits. Oxidative stress leading to neuronal cell death is a central process involved in the initiation and progression of neurodegenerative diseases and aging and, as such, is considered a potential target for the treatment of these diseases [1, 2]. Reactive oxygen species (ROS)-elicited oxidative stress is one of the major factors involved in the aging process and may serve as one of the mechanisms underlying age-related degenerative diseases [3]. Indeed, increased ROS production damages almost every intracellular macromolecule in neurons, including proteins, lipids, and DNA, resulting in cellular dysfunction, mutations, and/or cell death. Therefore, the regulation of ROS levels is important to prevent or treat neurodegenerative diseases and aging. In recent years, natural dietary components with antioxidant activity have attracted much attention due to their role in modulating oxidative stress associated with brain aging and chronic conditions [4, 5]. H₂O₂ is a key metabolite in oxidative stress which occurs in normal metabolism in mammalian cells at concentrations ~10 nM [6]. Higher concentrations lead to adaptive stress responses via nuclear factor erythroid 2-related factor 2 (Nrf2)/Keap1, while supraphysiological concentrations of H₂O₂ (>100 nM) lead to damage of biomolecules [6]. H₂O₂ was also shown to modulate the activity of the transcription factor cyclic AMP response element-binding protein (CREB) [7].

In a previous study, it was shown that an infusion prepared from the desert plant Pulicaria incisa (Lam.) DC. (Pi) protected astrocytes from oxidative stress-induced cell death, attenuated the induced ROS levels, demonstrated antioxidant properties in cell-free assays, and induced the transcription of glial-derived neurotrophic factor (GDNF) [8, 9]. The infusion also triggered anti-inflammatory activity and inhibited the activation of microglial cells which secrete ROS [9]. Infusions of Pulicaria incisa are used in traditional medicine for treating heart diseases; the species also provides a tea which is used by Bedouins [10].

### GENERAL DESCRIPTION

In one aspect, the present invention provides a pharmaceutical or nutritional composition for preventing, attenuating, or ameliorating the symptoms of neuronal cell death in a subject, wherein the composition comprises an infusion of the plant Pulicaria incisa (Pi) and one or more physiologically acceptable carriers.

In another aspect, the present invention provides a pharmaceutical or nutritional composition for reducing anxiety, wherein the composition comprises an infusion of the plant Pulicaria incisa (Pi) and one or more physiologically acceptable carriers.

In another aspect, the present invention provides an infusion of the plant Pulicaria incisa (Pi) for use in a method of preventing, attenuating, or ameliorating the symptoms of neuronal cell death in a subject in need thereof, the method comprising administering to the subject a therapeutically effective amount of said infusion of the plant Pulicaria incisa (Pi).

In another aspect, the present invention provides an infusion of the plant Pulicaria incisa (Pi) for use in reducing anxiety in a subject in need thereof, the method comprising administering to the subject a therapeutically effective amount of said infusion of the plant Pulicaria incisa (Pi).

In another aspect, the present invention provides a method of preventing, attenuating, or ameliorating the symptoms of neuronal cell death, wherein the method comprises administering to a subject in need thereof a therapeutically effective amount of an infusion of the plant Pulicaria incisa (Pi) or a composition comprising an infusion of the plant Pulicaria incisa (Pi).

In another aspect, the present invention provides method of reducing anxiety, wherein the method comprises administering to a subject in need thereof a therapeutically effective amount of an infusion of the plant Pulicaria incisa (Pi) or a composition comprising an infusion of the plant Pulicaria incisa (Pi).

In one embodiment, the subject has an age-related neuronal pathology.

In one embodiment, the age-related neuronal pathology comprises normal ageing of the brain.

In one embodiment said normal ageing of the brain is characterized by an increase in neuronal disfunction and/or a reduction in neuronal viability.

In one embodiment, said subject is an elderly subject.

In some embodiments, said composition is in the form of a food article, a beverage, a food additive, a food supplement, or herbal drug.

In other embodiments, said composition is in the form of tablets, capsules, liquid syrups, nasal spray, nasal drops, soft gels, suppositories, patches, and enemas.

In one embodiment, said composition is a pharmaceutical composition comprising a pharmaceutically acceptable carrier.

In some embodiments, said pharmaceutical composition is administered by oral, intraperitoneal, subcutaneous, transcutaneous, topical, intramuscular, intraarticular, subconjunctival, intranasal, or intraocular administration.

In one embodiment, said composition is administered at a concentration of between about 1 mg/kg and 100 mg/kg.

In one embodiment, said composition is provided orally at a concentration of between about 16 mg/kg and about 50 mg/kg.

In one embodiment, said composition is administered or consumed daily.

### BRIEF DESCRIPTION OF THE DRAWINGS

To better understand the subject matter that is disclosed herein and to exemplify how it may be carried out in practice, embodiments will now be described, by way of non-limiting example only, with reference to the accompanying drawings, in which:

FIG. 1A-B are GC/MS chromatograms of a metabolomic analysis of Pi infusion. Pi infusion was run on an SPE Sep-Pak C18 cartridge and eluted with water to yield the aqueous phase (1A), followed by methanol elution to yield methanolic phase (1B). Extracts were derivatized prior to analysis.

FIG. 2A is a graph showing viability of cells treated with increasing concentrations of hydrogen peroxide (H₂O₂). Twenty hours later, cell viability was measured using the XTT assay. The results of untreated cells were defined as 100% viability. 0.745 OD=100%. The results are presented as mean±SEM and represent one experiment (out of two experiments) with 6 repetitions. The results were analyzed by one-way ANOVA followed by the Tukey-Kramer multiple comparisons test. Different letters (A, B, C, D) represent statistically significant differences (p<0.001).

FIG. 2B is a graph showing viability of cells grown without (w/o) H₂O₂, in the presence of H₂O₂ with no treatment (H₂O₂ only) and cells subjected to increasing concentrations of Pi infusion before exposure to H₂O₂. The results are presented as mean±SEM and represent one experiment with 6 repetitions. The results were analyzed by one-way ANOVA followed by the Tukey-Kramer multiple comparisons test. Columns marked with different letters (A, B, C, D, E) represent statistically significant differences (p<0.05).

FIG. 2C is a graph showing the % viability of SH-SY5Y cells. Cells were treated with increasing concentrations of Pi infusions. The results of untreated cells (0) were defined as 100% viability. 0.364 OD=100%. The data are expressed as mean±SEM of two independent experiments (n=12). The results were analyzed by one-way ANOVA.

FIG. 3A-3B is a graph showing caspase 3 activity in fluorescence units (FU) measured for 30 minutes in cell lysates of untreated cells, cells treated with Pi infusion only, cells exposed to H₂O₂, and cells exposed to H₂O₂ and treated with 100 μg/ml or 200 μg/ml Pi infusion, as measured 3 hours (3A) and 20 hours (3B) after the induction of oxidative stress by H₂O₂. The results are presented as mean±SD of one experiment with two repetitions.

FIG. 3C is a graph showing caspase 3 activity (as % of untreated cells) in cell lysates as measured 20 h after the induction of oxidative stress for 30 min. The graph compares cells with exposure (“with H₂O₂”) and without exposure (“W/O H₂O₂”) to H₂O₂, that were treated with 100 μg/ml Pi infusion or kept untreated (0). The results are presented as mean±SEM and represent three independent experiments (n=6). The results were analyzed by one-way ANOVA followed by the Tukey-Kramer multiple comparisons test. Columns marked with different letters (A, B, C) represent statistically significant differences. (p<0.05).

FIG. 4 is a graph showing intracellular reactive oxygen species (ROS) levels (FU) in cells treated with increasing concentrations of Pi infusion (50 μg/ml, 100 μg/ml, or 200 μg/ml) for 2 hours prior to addition of H₂O₂ (200 μM), as compared to untreated cells without (w/o) H₂O₂. Fluorescence, indicating ROS levels, was measured 1 h and 18 h thereafter. The results are presented as mean±SEM and represent two independent experiments (n=12). The results were analyzed by one-way ANOVA followed by the Tukey-Kramer multiple comparison test. Columns marked with different letters (A, B, C, etc.) represent statistically significant differences (p<0.05).

FIG. 5A-C shows representative images of immunostaining for Nrf2 in untreated SH-SY5Y cells (A) and in SH-SY5Y cells treated with Pi infusion (200 μg/mL) for 1 h (B) or 2 h (C). The cells' nuclei were stained with DAPI. Bar: 20 μm.

FIG. 5D-F are graphs showing the intensity of nuclear Nrf2 staining (5D), cytosolic Nrf2 staining (5E), and total Nrf2 staining in the cell, in arbitrary units, as measured in 11 different photographed fields. Nrf2 intensity was normalized to DAPI intensity. The results are presented as mean±SEM (n=11) and were analyzed by one-way ANOVA followed by the Tukey-Kramer multiple comparison test. Columns marked with different letters (A, B) represent statistically significant differences (p<0.05).

FIG. 6 is a graph showing nuclear Nrf2 levels (OD) in SH-SY5Y cells pretreated with Pi infusion (200 μg/mL) for 2 h, and then incubated for an additional 2 h or 18 h with H₂O₂ (200 μM) (total incubation of 4 hours or 20 hours), as compared with untreated cells or with cells that were treated with Pi infusion without exposure to H₂O₂. The results are from a representative experiment out of two independent experiments, with each experiment conducted in duplicates. The results are presented as mean±SEM (n=4) and were analyzed by one-way ANOVA followed by the Tukey-Kramer multiple comparison test. Columns marked with different letters (A, B, C) represent statistically significant differences (p<0.05).

FIG. 7 is a graph showing levels of phosphorylated CREB (pCREB) in lysates of cells that were pretreated for 2 h with increasing concentrations of Pi infusion (50, 100 or 200 μg/ml), with or without (w/o) subsequent exposure to H₂O₂ (200 μM). The results are presented as mean±SEM of three experiments performed in duplicates (n=6) and were analyzed by one-way ANOVA followed by the Tukey-Kramer multiple comparison test. Columns marked with different letters (A, B) represent statistically significant differences p<0.001.

FIG. 8A-B are graphs showing the percentage of pyknotic neurons, calculated as the number of pyknotic neurons out of the total number of neuronal cells (vital+pyknotic) (8A) and number of vital cells (8B), in the cornu Ammonis 3 (CA3) and dentate gyrus (DG) regions of hippocampus of brains of mice treated daily for 18 months with Pi infusion, 200 mg/kg or 600 mg/kg, as compared to untreated mice. The results are presented as mean±SEM (n=9). Columns marked with different letters (A, B, C) represent statistically significant differences p<0.05.

FIG. 9 is a graph showing time spent in the interior area of the arena (the center zone) in seconds (sec) by mice in the control group and by mice in the group treated with either 200 mg/kg Pi or 600 mg/kg Pi, for 8 or 10 months. The results are presented as the mean±S.E.M. Statistical analyses were performed using One Way ANOVA following HSD Tukey. At the age of 8 months: n=20, ***p=0.04. At the age of 10 months: n=17, ***p=0.02.

### DETAILED DESCRIPTION OF EMBODIMENTS

The present invention is based on the surprising finding that an infusion prepared from the plant Pulicaria incisa (also referred to herein as Pi infusion) has neuroprotective effects on cultured neurons and in aging mice. The inventors explored the effects of Pi infusion in a hydrogen peroxide (H2O2)-induced oxidative stress model in SH-SY5Y human neuroblastoma cells, which serve as an in vitro system to study neuronal cells. H₂O₂-induced ROS accumulation and caspase 3 activity decreased SH-SY5Y viability. These deleterious effects could be prevented by pretreatment of cells with Pi infusion. Additionally, the Pi infusion upregulated cellular levels and nuclear translocation of nuclear factor erythroid 2-related factor 2 (Nrf2) as well as the phosphorylation of cyclic AMP response element-binding protein (CREB). Aging mice treated daily for 18 months with Pi infusion exhibited reduced neuronal cell death in the hippocampus as compared to age-matched controls.

The present invention thus provides therapeutic methods and uses of Pi infusion for treating neuronal degeneration and ageing in the brain.

Furthermore, since the effects of the Pi infusion were evident upon oral administration to the mice, the present disclosure further encompasses administration of the Pi infusion as a nutritional supplement or functional food/beverages.

Particularly, the Pi infusion may be administered as a beverage (e.g., herbal infusion, tea, nutritional shake, or juice) suitable for daily consumption.

Therefore, in a first of its aspects, the present invention provides a pharmaceutical or nutritional composition for preventing, attenuating, or ameliorating the symptoms of neuronal cell death in a subject, wherein the composition comprises an infusion of the plant Pulicaria incisa (Pi) and one or more physiologically acceptable carriers.

As used herein the term “pharmaceutical composition” refers to a composition comprising an infusion of the plant Pulicaria incisa (Pi), wherein said composition is provided as a medicament. As used herein the term “nutritional composition” refers to a composition comprising an infusion of the plant Pulicaria incisa (Pi), wherein said composition is provided as a food supplement, nutrition additive, food, or beverage.

As used herein the term “Pulicaria incisa (PJ)” refers to a desert plant that belongs to the Asteracea family. The term refers both to wild-growing Pi, namely to uncultivated Pi plants that were not irrigated by human cultivators, as well as to cultivated Pi plants that are grown for agricultural purposes in an enclosed area of land (e.g., a field) and are irrigated by human cultivators.

The terms “infusion of the plant Pulicaria incisa (Pi)” or “Pulicaria incisa (Pi) infusion” are used interchangeably herein and refer to an infusion of the aerial parts of Pi plants. The aerial parts of the plant include stems, leaves, and flowers. The infusion can be prepared using any method known in the art, for example by soaking dried aerial Pi parts in boiling water and removing the solid parts, e.g., by centrifugation and filtration. A non-limiting example of a method of preparing the Pi infusion is provided in the Examples below. The infusion can also be prepared from a freshly collected plant. As used herein, the term Pi infusion also encompasses a dried form of the infusion, for example a powder that can be incorporated into food products or beverages.

Profiling of the infusion by gas chromatography-mass spectrometry revealed multiple components and identified chlorogenic acid, quercetin, and aucubin as some of its main constituents. A non-limiting example of the Pi infusion content is provided in Example 1, Table 1.

The terms “preventing”, “attenuating”, or “ameliorating the symptoms” is used conventionally and refers to the management or care of a subject for the purpose of combating, alleviating, reducing, relieving, or improving a subject's neuronal pathology, or any symptom thereof. In one embodiment, the subject suffers from an age-related neuronal pathology. The term also encompasses prophylactic treatment of subjects at risk of neuronal pathologies, e.g., elderly subjects, or subjects with a genetic predisposition. The terms encompass any reduction in the subject's neuronal pathology, or any symptom thereof as evidenced, for example, by measurement of cognitive, physiological, radiological (e.g., by Magnetic Resonance Imaging (MRI)), and/or clinical indicators of a neuronal pathology.

As used herein the term “neuronal cell death” refers to a reduction in the viability of neurons, particularly in the brain, for example, but not limited to, the hippocampus. The neuronal cell death can be associated with age-related neuronal pathologies or associated with neurodegenerative diseases.

As used herein the term “age-related neuronal pathologies” refers to age-related disorders of the central nervous system (CNS), particularly disorders that are associated with neuronal disfunction, or reduction in neuronal viability (neuronal loss). In one embodiment the term encompasses age-related neurodegenerative diseases. In one embodiment the term encompasses normal ageing of the brain. Normal ageing of the brain is associated with increased levels of pathology leading to cognitive decline in the elderly. Normal ageing of the brain may also lead to Alzheimer's disease.

The term “elderly subject” may encompass for example subjects aged 65 years, or more.

As shown in the Examples below, treatment of ageing mice with the Pi infusion of the invention resulted in a remarkable decrease in anxiety-like behavior.

Accordingly, the present invention also provides a pharmaceutical or nutritional composition for reducing anxiety in a subject, wherein the composition comprises an infusion of the plant Pulicaria incisa (Pi) and one or more physiologically acceptable carriers.

In one embodiment, the subject is an elderly subject.

As used herein the term “reducing” refers to any amelioration, elimination, remedy or slowing down of anxiety in a subject. Assessment of anxiety level and reduction in anxiety can be tested using assays well known in the art, for example the Zung self-rating anxiety scale, Hamilton Anxiety Scale (HAM-A), Beck Anxiety Inventory (BAI), Social Phobia Inventory (SPIN), Penn State Worry Questionnaire, and Generalized Anxiety Disorder Scale.

The compositions of the invention may be administered as such, or may be incorporated into food products, beverages (e.g., juices, herbal infusions, nutritional shakes, or tea) or combined with commonly used food additives such as corn syrup. The composition may be incorporated into a nutritional beverage provided to subjects in need of nutritional complementation. The composition may also be provided as a dry powder for mixing in a food product or beverage.

In one embodiment, the composition of the invention is a nutritional composition administered by feeding.

In one specific embodiment, the composition of the invention is administered as a beverage.

In other embodiments, the composition of the invention is a pharmaceutical composition that can be administered and dosed in accordance with good medical practice. For example, the pharmaceutical composition can be introduced to the body by any suitable route including oral, intraperitoneal, subcutaneous, transcutaneous, topical, intramuscular, intraarticular, subconjunctival, or mucosal, e.g., intranasal, or intraocular administration. The compositions may be administered systemically or may be locally administered. Local administration may be facilitated by using an implant that acts to retain the active dose at the site of implantation. The active agent may be formulated for immediate activity, or it may be formulated for sustained release.

In yet some further embodiments, the composition of the invention may optionally further comprise at least one of pharmaceutically acceptable carrier/s, excipient/s, additive/s diluent/s and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, formulations are prepared by uniformly and intimately bringing into association the active ingredients of the invention with liquid carriers or finely divided solid carriers or both, and then, if necessary, shaping the product.

The compositions may be formulated into any of many possible dosage forms such as, but not limited to, tablets, capsules, liquid syrups, nasal spray, nasal drops, soft gels, suppositories, patches, and enemas.

The Pi infusion may be diluted to a desired final concentration by mixing with an appropriate vehicle solution, e.g., water, a hydrophilic solution, or ethanol.

In addition to the ingredients particularly mentioned above, the formulations may also include other agents conventional in the art having regard to the type of formulation in question.

Still further, the compositions of the invention and any components thereof may be applied as a single daily dose or multiple daily doses.

In one embodiment, the “daily dose” refers to one administration of Pi infusion per day. In other embodiments, the “multiple daily doses” refers to two or more administrations of Pi infusion per day.

The compositions may be administered once symptoms of age-related neuronal pathology or anxiety become evident.

In another embodiment, the compositions are administered as a prophylactic treatment for elderly subjects or to subjects that are prone to suffer from anxiety.

In another embodiment, the compositions are administered for an unlimited time-period (also referred to as chronic administration), for example for one year, two years or more, or throughout the subject's lifespan. For example, the compositions may be given as a food supplement or a beverage for regular consumption.

The composition of the invention can be administered alone, or in combination with other active agent(s). In certain embodiments the additional active agents include, but are not limited to, 3,5,4′-trihydroxy-6,7,3′-trimethoxyflavone (TTF), Geranium oil, Chlorogenic acid derivatives and caffeic acid derivatives.

The compositions of the present invention may be combined with other types of treatments including behavioral therapy, rehabilitation therapy, diet restrictions and pharmacological intervention.

In another aspect, the present invention provides a method of treating, preventing, or ameliorating the symptoms of age-related neuronal pathologies, wherein the method comprises administering to a subject in need thereof a therapeutically effective amount of an infusion of the plant Pulicaria incisa (Pi) or a composition comprising an infusion of the plant Pulicaria incisa (Pi).

By the term “administering”, it is meant that the composition is delivered to a subject by any means or route which is effective to achieve the desired result, including e.g., oral, parenteral, enteral, intraperitoneal, topical, transdermal (e.g., using any standard patch), subcutaneous, intravenous, intra-arterial, intramuscular, buccal, sublingual, ophthalmic, nasal, by aerosol, by inhalation, rectal, vaginal, and intrathecal.

In some embodiments the composition of the invention is administered to human subjects in an amount of between about 1 mg/kg to 100 mg/kg per day, depending upon the subject's physical condition, the severity of disease, mode of administration, etc. In specific embodiments the composition of the invention is orally administered to human subjects in an amount of about 16 mg/kg (which approximately corresponds to an amount of 200 mg/kg in mice) or about 50 mg/kg (which approximately corresponds to an amount of 600 mg/kg in mice) per day.

The compositions can be administered at any suitable time, e.g., prior to or after a meal, prior to activity, prior to sleeping and at different times of the day, e.g., in the morning, in the evening etc.

### EXAMPLES

### Materials and Methods

### Plant Material and Preparation of Pi Infusion

The Pi plants were grown in Southern Arava Research and Development, Hevel Eilot, Israel. Aerial parts of Pi plants were air-dried. The Pi infusion was prepared by soaking dried aerial Pi parts in a beaker containing boiling autoclaved double distilled water (DDW) in a ratio of 1 g plant:20 mL DDW. The beaker was allowed to cool at room temperature (RT) for 45 min, and the infusion was transferred into tubes. The tubes ware centrifuged (4000 RPM, 10 min, RT), the supernatant was collected, filtered through 0.2 μm filters, and aliquots were frozen and stored at −20° C., until use. To determine the concentration of Pi infusion, a 40 mL sample of the filtered supernatant was lyophilized to obtain a powder that was weighed. The average concentration of the various preparations of Pi infusion was ~7 mg/mL. For in vivo experiments in mice, Pi infusions were prepared similarly, except that sterile autoclaved tap water was used instead of DDW.

### Chemical Characterization of Pi Infusion

### Sample Preparation

The infusion was lyophilized (Alpha-Christ, Christ, Osterode am Harz, Germany) and stored at −20° C. Samples (30 mg) and 50 μl of 0.2 mg/mL ribitol (Sigma Aldrich, St. Louis MO, USA) in methanol (Arcos, NJ, USA) as internal standard, were dissolved in 0.2 ml DDW (Synergy® UV Water Purification System, Merck, Darmstadt, Germany). A SEP-PAK C18 cartridge (Waters, Milford, MS, USA) was pre-washed with 1 ml methanol, followed by 1 mL H₂O. The sample was loaded on the cartridge and eluted with 1.5 mL DDW. The aqueous phase was collected. Next, the cartridge was washed with 1.5 ml methanol, and the methanolic phase was collected. A 0.5 mL sample of each phase was lyophilized and then derivatized, according to an established protocol (Mahajna, S., et al., Molecules, 2019. 24(22)).

### Silylation Derivatization

Briefly, 70 μL methoxyamine hydrochloride (Sigma, Aldrich, St. Louis MO, USA) solution (40 mg/mL in pyridine) was added to each lyophilized sample in an Eppendorf tube. Samples were then incubated in a thermoshaker for 90 min, after which, 100 μL N-methyl-N-(trimethylsilyl)-trifluoroacetamide (MSTFA; Sigma, Aldrich, St. Louis MO, USA) were added. Samples were mixed in a thermoshaker at 30° C. for 30 min, and 70 μL were used for injection into GC/MS.

### Gas Chromatography-Mass Spectrometry Analysis

GC/MS analysis was performed with an Agilent 6850 GC, equipped with Agilent 5975C single quadrupole MS, CTC-PAL RSI 85 auto-sampler, and HP-5MS capillary column (0.25 μm×30 m×0.25 mm). The following conditions were applied: injector temperature 250° C., initial temperature 50° C. for 5 min, a gradient of 5° C./min until 180° C., a gradient of 10° C./min until 270° C. and a hold time of 10 min, and increasing to 320° C. The MS parameters were set as follows: source temperature 230° C., transfer line 325° C., quadrupole: 150° C., detector 325, positive ion monitoring and electron ionization (EI)-MS measurement at 70 eV (Mahajna, S., et al., Molecules, 2019. 24(22)). Helium was used as a carrier gas, infused at 0.6 mL/min.

### Data Processing and Compound Annotation

Data were analyzed by the Unknown Analysis software (Agilent, Santa Clara, CA, USA). The percentage composition of the samples was computed from the GC peak areas. Library searches were conducted using the National Institute of Standards and Technology (NIST) 14 GC/MS Library and mass spectra from the literature. Component-relative percentages were calculated based on GC peak areas without using correction factors. Commercially available standards were used for 2-hydroxybenzoic acid, 4-hydroxybenzoic acid, genistic acid, protocatechuic acid, quininic acid, gallic acid, ferulic acid, caffeic acid, (2R, -E) catechin, and quercetin (Sigma, Aldrich, St. Louis MO, USA).

### Cell Growth

SH-SY5Y (ATCC CRL-2266) were grown in a 1:1 mixture of Eagle's minimum essential medium (EMEM) (ATCC, VA USA) and F12 medium (Biological Industries, Israel), supplemented with 10% fetal bovine serum (FBS) and 1% penicillin-streptomycin (Biological Industries, Israel). Passages 12-20 were used in this study. All the experiments included a vehicle control group containing double distilled water (DDW) instead of the Pi infusion.

### Measurement of Cell Viability

SH-SY5Y cells were seeded (30,000/well) in a 96-well plate and were grown for 24 h in a 1:1 mixture of EMEM:F12 medium, containing 2% FBS and 1% penicillin-streptomycin. The medium was replaced the next day with fresh medium containing different concentrations of Pi infusion, and cells were incubated for 2 h. Then, cells were treated with H₂O₂ (200 μM), and cytotoxicity was measured 20 h later. Cytotoxicity was measured using the 2,3-bis-(2-methoxy-4-nitro-5-sulfophenyl)-2H-tetrazolium-5-carboxanilide (XTT) assay, as per the manufacturer's instructions (Sartorius AG, Goettingen, Germany). Absorbance was measured at 490 nm in a Synergy2 multi-detection microplate reader (BioTek Instruments, Inc., Winooski, VT, USA). The absorbance of the plate before the addition of XTT served as the background absorbance.

### Quantitation of Nrf2 Levels in Nuclear Extracts

SH-SY5Y cells were seeded (10×10⁶/25² cm flasks) and cultured overnight in a 1:1 mixture of EMEM:F12 medium, containing 2% FBS and 1% penicillin-streptomycin. The medium was replaced the next day with a fresh medium. The cells were pretreated with Pi infusion, after which, 200 μM H₂O₂ was added for an additional 2 h or 18 h. Nuclear proteins were extracted using a nuclear extraction kit (Cayman Chemical, MI USA), according to the manufacturer's instructions, and protein concentrations were determined using the BCA protein assay kit (Pierce Biotechnology, IL USA). Equal amounts (20 μg protein) from each sample were tested (27° C. in a heated block) for Nrf2 levels using the Nrf2 Transcription Factor Assay kit (Cayman Chemical, MI USA). This assay kit detects specific transcription factor DNA-binding activity in nuclear extracts. A specific dsDNA sequence containing the Nrf2 response element is immobilized onto a 96-well plate. Nrf2 from nuclear extracts specifically binds to the Nrf2 response element and is detected by the addition of a specific primary anti-Nrf2 antibody. A secondary antibody conjugated to HRP is added to provide a sensitive colorimetric readout at 450 nm.

### Immunocytochemistry

SH-SY5Y cells were replated (150,000 cells/well) into 24-well plates containing glass coverslips, in a 1:1 mixture of EMEM:F12 medium, containing 2% FBS and 1% penicillin-streptomycin. The medium was replaced the next day with fresh medium, and cells were then pretreated with Pi infusion for either 1 h or 2 h. Then 200 μM H₂O₂ was added for either 2 h or 18 h. Following treatment, the cells were washed with warm phosphate buffered saline (PBS) and then fixed with 4% formaldehyde for 15 min at room temperature, washed 3 times with PBS, and permeabilized for 3 min with 0.01% Triton-x in PBS. The cells were washed again and incubated with 10% normal mouse serum in PBS, for 30 min, at room temperature, then rinsed with PBS and incubated overnight, at 4° C., with Alexa Fluor 488-conjugated mouse monoclonal anti-human Nrf2 antibody (sc-518033, Santa Cruz Biotechnology, Dallas, Texas, USA) in PBS containing 1.5% normal mouse serum. To visualize the nuclei, the cells were washed again, stained with 4′,6-diamidino-2-phenylindole (DAPI) 1:10000 in PBS (15 min, at room temperature), and rinsed 4 times with PBS. Images were acquired by a Leica SP8 laser scanning microscope (Leica, Wetzlar, Germany), equipped with solid-state lasers with 405 and 488 nm light, HC PL APO CS 63×/1.2 water immersion objective (Leica, Wetzlar, Germany), and Leica Application Suite X software (LASX, Leica, Wetzlar, Germany). DAPI and Alexa Fluor 488 emission signals were detected with PMT and HyD (hybrid) detectors in ranges of 415-490 and 500-550 nm, respectively. An average of 11 images/coverslip were captured, with the same exposure time for all samples. The mean fluorescence values in regions of interest were quantified with ImageJ software (NIH).

### Determination of Caspase-3 Activity

SH-SY5Y cells were re-plated into 6-well plastic plates at a density of 2×10⁶ cells/2.5 mL/well in 2.5 ml of a 1:1 mixture of EMEM:F12 medium, containing 2% FBS and 1% penicillin-streptomycin. The medium was replaced the next day with fresh medium and cells were treated with Pi infusion, for 2 h. Then, 200 μM H₂O₂ was added to each well, and cells were incubated for 20 h before being scraped into tubes and washed twice with 1 mL cold PBS (125 g, 5 min). The supernatant was discarded, and pellets were incubated in a cold lysis buffer comprised of 10 mM TRIS pH 7.5, 100 mM NaCl, 1 mM EDTA, and 0.01% Triton X-100, for 30 min, on ice. After freezing in liquid nitrogen and thawing, the supernatant was centrifuged (10,000 g, 15 min) and the lysates were frozen at −80° C. Protein concentration was determined using a BCA kit. Equal amounts of protein (20 μg) were tested for caspase-3 activity using the EnzChek Caspase-3 assay kit (Molecular Probes, OR USA). Fluorescence was measured using the Synergy™ Neo2, Multi-Mode microplate reader (BioTek Instruments, Inc., Winooski, VT, USA)

### Determination of Phospho-CREB Levels

SH-SY5Y cells were re-plated into 6-well plates at a density of 2×10⁶ cells/2.5 mL/well in a 1:1 mixture of EMEM:F12 medium, containing 2% FBS and 1% penicillin-streptomycin. Medium was replaced the next day with fresh medium, and cells were treated with Pi infusions at the indicated concentrations, for 2 h. Thereafter, 200 μM H₂O₂ was added for 30 min. Medium was collected into tubes and cells were washed with cold PBS and then lysed on ice in 0.5 mL/well lysis buffer containing 20 mM Tris-HCl (pH 7.5), 150 mM NaCl, 1 mM Na₂ EDTA, 1 mM EGTA, 1% Triton x-100, 20 mM sodium pyrophosphate, 25 mM sodium fluoride, 1 mM β-glycerophosphate, 1 mM Na₃VO₄, 1 μg/ml leupeptin and 1 mM PMSF. After 5 min, cells were scraped and collected into tubes, frozen in liquid nitrogen, thawed, and centrifuged for 10 min at 13,000 g, 4° C. The supernatant was collected and stored at −80° C. Protein concentration was determined using the BCA kit protein assay kit (Pierce Biotechnology, IL USA). Equal amounts of protein were analyzed using the PathScan Phospho-CREB sandwich ELISA kit (Cell Signaling Technology, MA USA), according to the manufacturer's instructions.

### Evaluation of Intracellular ROS Levels

Intracellular ROS levels were determined using the non-fluorescent cell-permeable compound, 2′7′-dichlorofluorescein diacetate (DCF-DA; Sigma, Aldrich, St. Louis MO, USA). SH-SY5Y cells were plated onto 24-well plates (30,000 cells/0.5 mL/well) and incubated for 24 h in a 1:1 mixture of EMEM:F12 medium, containing 2% FBS and 1% penicillin-streptomycin. Cells were then labeled with DCF-DA (20 PM) for 30 min, at 3 7° C. Following incubation with DCF-DA, cultures were rinsed twice with PBS which was then replaced with fresh medium. ROS levels (fluorescence) at time zero were measured in a plate reader with excitation at 485 nm and emission at 520 nm. SH-SY5Y cells were then treated with Pi infusions at the indicated concentrations, for 2 h, before H₂O₂ was added. Fluorescence intensities (indicative of ROS levels) were measured 1 h and 18 h later, by the Synergy™ Neo2, Multi-Mode microplate reader (BioTek Instruments, Inc., Winooski, VT, USA). The background fluorescence intensities at time zero were subtracted from the intensities at 1 h and 18 h.

### In Vivo Establishment of the Aging Mouse Model

The research was conducted following The National Institute of Health (NIH) guidelines for the care and use of laboratory animals and was approved by The National Permit Committee for animal science (IL-20-2-60). Twenty-seven female C57BL mice (5-week-old, weighing ~15.5 g) were purchased from Envigo, Israel. The mice were maintained under a controlled 12 h light/12 h dark cycle, with free access to food and water. The mice were randomly allocated into three groups (9 mice/group). Control mice drank only water, without Pi infusion. Nine mice received a daily Pi infusion (200 mg/kg or 600 mg/kg body weight) in the drinking water for 18 months, starting from −1.5 months of age. The Pi infusions were prepared as described above, except that instead of DDW, they were prepared in sterile tap water. Each day, a new frozen tube of Pi infusion was thawed and then diluted into autoclaved tap water, which was added to the drinking autoclaved tap water bottles to a final volume of 60 mL. Each bottle was covered with aluminium foil to minimize exposure to light.

### Hematoxylin and Eosin Staining Assay

At 19 months of age, mice were sacrificed, and brains were harvested and stored for 48 h in 4% formaldehyde. The tissues were processed for paraffin embedding, and serial sections (4 μM thick) were cut off with a microtome. Paraffin-embedded sections were deparaffinized and rehydrated and then stained with hematoxylin & eosin (H&E) following a previously published method (Min et al., Chem Biol Interact, 2015. 242: p. 153-62). After H&E staining, histopathological changes were then assessed as described below.

### Digital Morphometry and Histopathological Evaluation of Pyknosis in the Hippocampus

The number of hippocampal neurons at the cornu Ammonis 3 (CA3) and dentate gyrus (DG) regions was quantified at a magnification of ×20, using MATLAB software by brightness, color, and morphological-based segmentation. The image analysis system used was Image Pro Ver. 10.0.11 (Media Cybernetics). Identification of pyknosis in the hippocampus was based on the color and brightness of pixels, as measured using the ‘Smart Segmentation’ option (Machine Learning option) combined with the “Classification learning” process, which involves teaching the system morphometric and density parameters indicative of vital versus pyknotic cells (Reedy et al., Materials Research Society Symposium Proceedings, 2014. 1656). This selection is saved and applied to each new image and can be changed if needed (the software keeps learning the changes). The percentage of the pyknotic neurons was calculated as the number of pyknotic neurons out of the total number of neurons (vital+pyknotic).

### Open Field Test

Open field tests were performed with 8- and 10-months old mice. Before the test, the mice were habituated to the experimental space. The habituation was 30 minutes long, during which the mice stayed in their home cages. The experiments were carried out during the light cycle of the mice under white light. Distance traveled and time spent in the center zone by each animal was recorded using the EthoVision XT (Noldus Information Technology, Wageningen, The Netherlands) video tracking system and were analyzed using Ethovision 14XT (Noldus) software. Each mouse was placed alone in a rectangular arena (50 cm×50 cm, 40 cm high, made from plastic material) for 15 minutes. A standard measure is the amount of time the animal spent walking along the exterior edge vs. the interior; the more anxious the animal is, the more time it would spend on the exterior edge of the maze.

### Statistical Analyses

Statistical analyses were performed with one-way ANOVA followed by Tukey-Kramer multiple comparison tests, using the JMP 7 statistical analysis software program (SAS Institute, Inc., Duxbury, USA). In this analysis, columns with different letters (i.e., A, B, C) are significantly different (p<0.05), and columns with the same letter are not significantly different (p>0.05).

### Example 1

### Chemical Characterization of Pi Infusions

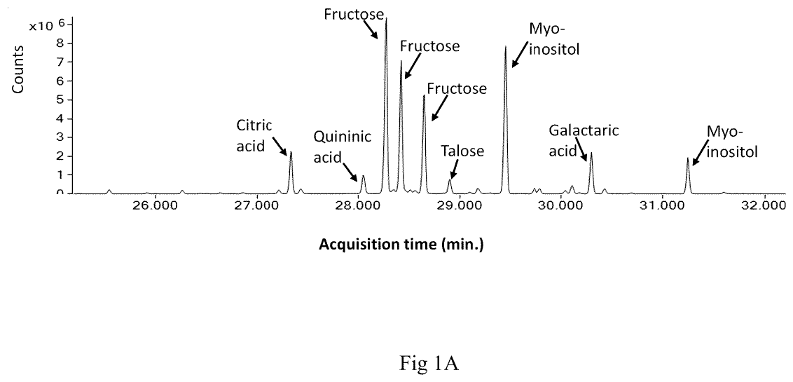

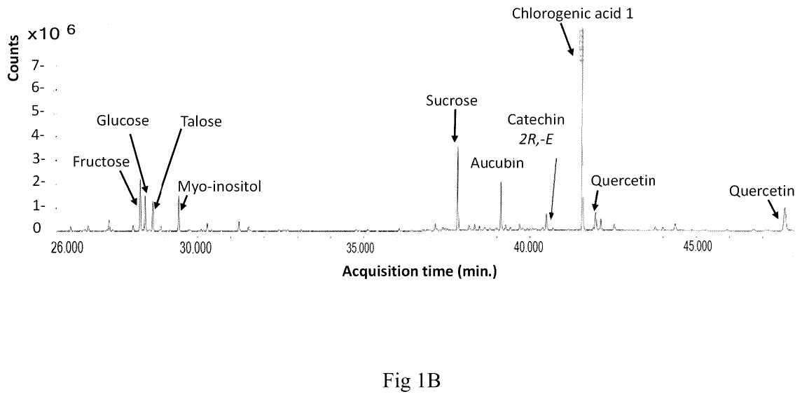

Pi infusions presented a rich metabolomic profile, comprised of a total of 88 compounds, including 13 organic acids (accounting for 6.5% of the total peak area of the methanolic phase), 4 alcohols (0.48%), 11 amino acids (0.97%), 26 sugars and saccharides (40.4%), 3 fatty acids (1.45%), 3 terpenes (8.37%), 35 phenolic compounds (34.8%) and 5 other compounds (6.8%). The main compounds in the Pi infusion and their relative contents in the methanolic and aqueous phases are presented in Table 1, and respective chromatograms are brought in FIG. 1. In the methanolic phase saccharides including sucrose (15.35% of total peak area), fructose (11.65%), myo-inositol (6.42%) and talose (4.52) were most abundant, while chlorogenic acid and its derivatives (17.77%), quercetin and its derivatives (8.08%), and aucubin (2.79%) were the most abundant non-saccharides. Saccharides were also the most abundant group of compounds in the aqueous phase, mainly fructose (25.91%), myo-inositol (25.90%), talose (10.31%), and glucose (9.04%), in addition to citric acid (3.81%), galactaric acid (3.57%) and malic acid (1.92%) as the prevalent non-saccharides. The analysis revealed the presence of various chlorogenic acid isomers. Phenolic acids rarely appear in a free form, and typically are esterified with quinic or tartaric acid or a sugar moiety at different locations, giving rise to various structural isomers (Kumar and Goel, Biotechnol. Rep (Amst), 2019. 24: p. e00370). Specifically, chlorogenic acid is a group of caffeic and quinic acid esters, comprising six different isomers (3-O-caffeoylquinic acid (3CQA), 4CQA, 5CQA, 3,4-dicaffeoylquinic acid (3,4-DQA), 3,5-DQA and 4,5-DQA), or 26 compounds when considering cis-trans isomerization (Clifford et al., Food Chemistry, 2008. 106(1): p. 379-385). These have also been reported to vary in occurrence, bioavailability, and bioactivity (Liang and Kitts, Nutrients, 2015. 8(1)). Similarly, flavonoids, e.g., quercetin, present characteristic versatility due to structural variability, owing to conjugation with various sugar moieties at various positions (Abu-Reidah, et al., Food Research International, 2017. 100: p. 494-500). As GC/MS metabolomic analysis cannot be used for reliable quantification, the current data only provided relative quantification of these compounds.

TABLE 1
Main compounds identified in the methanolic and
aqueous phases of Pulicaria incisa infusion.

| | | Total peak | Total peak |
|---|---|---|---|
| | | area (%) in | area (%) in |
| | | the methanolic | the aqueous |
| Compound | Rt | phase | phase |
| Malic acid | 21.884 | | 1.925 |
| 3-Aminophenol | 25.702 | 1.414 | |
| Citric acid | 27.333 | 1.034 | 3.806 |
| Quininic acid | 28.051 | | 1.754 |
| Fructose | 28.259 | 11.647 | 25.913 |
| Talose | 28.634 | 4.517 | 10.309 |
| Glucose | 28.646 | | 9.038 |
| Myo-Inositol | 29.433 | 6.418 | 25.897 |
| Quinoline, 5-chloro- | 29.442 | | 1.825 |
| 8-ethoxy-7-iodo- | | | |
| Galactaric acid | 30.296 | | 3.575 |
| Caffeic acid | 31.520 | 1.327 | |
| Sucrose | 37.814 | 15.348 | 8.261 |
| Turanose | 38.323 | | 1.240 |
| Aucubin | 39.131 | 2.792 | |
| Catechine (2R-E) | 40.487 | 2.115 | |
| Chlorogenic acid 1 | 41.573 | 16.192 | |
| Quercetin | 41.974 | 8.082 | |
| Chlorogenic acid 2 | 42.115 | 1.424 | |

Metabolites were analyzed by GC/MS metabolomics following C18 SPE extraction with methanol (methanolic phase) and DDW (aqueous phase). Presented are metabolites that comprise at least 1% of the total ion count (total peak area) of the respective chromatograms. Quercetin and Quininic acid were identified by commercial standards. Fructose and Quercetin represent the sum of numerous derivative peaks, resulting from N-methyl-N-(trimethylsilyl)-trifluoroacetamide (MSTFA) derivatization at different locations of the molecules. Chlorogenic acids 1 and 2 may represent different isomers, or different derivatives of the same isomer.

### Example 2

### Pi Infusion Protects Neuronal Cells Against Oxidative Stress-Induced Cell Death

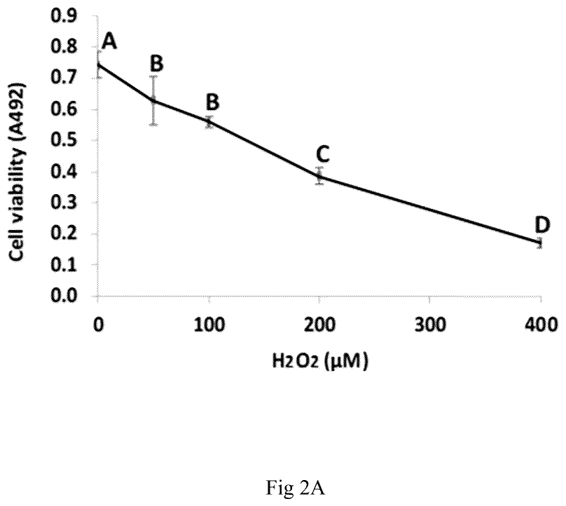

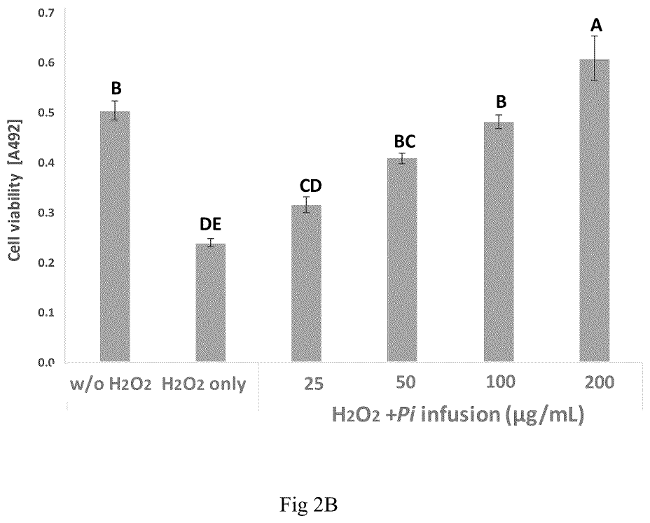

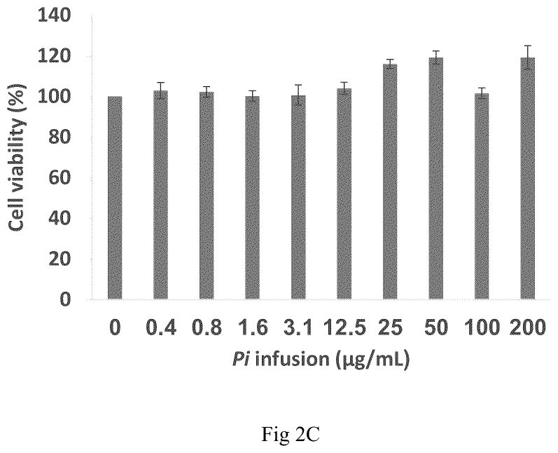

To determine the effects of Pi infusion on H₂O₂-induced neuronal cell death, the cytotoxicity of H₂O₂ in SH-SY5Y cells was first assessed using the XTT assay. Exposure of SH-SY5Y cells to H₂O₂ resulted in time- and concentration-dependent cell death (FIG. 2A). The concentration of H₂O₂ selected for subsequent experiments (200 μM) resembled the concentration reported in rat striatum under ischemic conditions [11]. Next, SH-SY5Y cells were treated with increasing concentrations of Pi infusions 2 h before exposure to 200 μM hydrogen peroxide. Twenty hours after the induction of the oxidative stress, cell viability was measured using the XTT assay. Apparently, pretreatment of the cells with Pi infusion before the induction of oxidative stress resulted in marked and statistically significant dose-dependent protection from cell death (FIG. 2B). To test for potential toxic effects of the Pi infusion, SH-SY5Y cells were treated with increasing concentrations of Pi infusions. Twenty-four hours later, cell viability was measured using the XTT assay. No significant differences were observed, and no Pi infusion cytotoxicity was recorded at any concentration tested (FIG. 2C).

### Example 3

Pi Infusion Inhibits H₂O₂-Induced Caspase-3 Activity

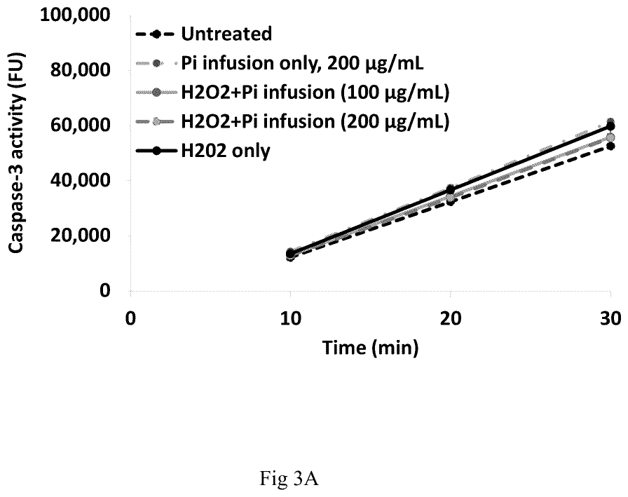

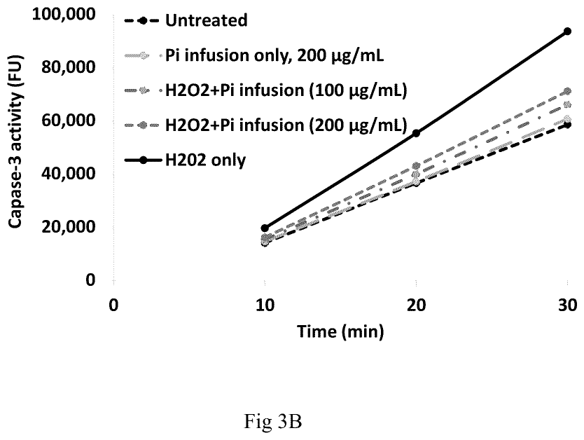

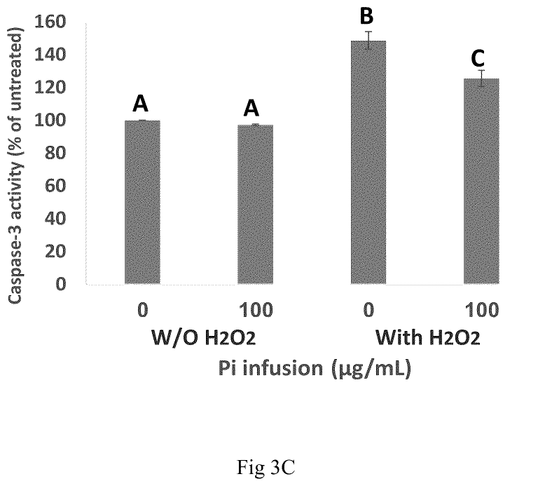

To determine whether inhibition of caspase 3 activity is involved in the neuroprotective mechanism of Pi infusion, SH-SY5Y cells were again pretreated with Pi infusion 2 h before exposure to H₂O₂. The enzymatic activity of caspase 3 in cell lysates was then determined 3 h and 20 h after exposure to H₂O₂. Extended exposure to H₂O₂ led to a 1.6-fold increase in caspase-3 activity relative to untreated cells, while preincubation with 100 μg/mL Pi infusion prior to H₂O₂ exposure inhibited this activity by 79% (FIG. 3A, 3B). After a 3-h exposure to H₂O₂, only a minor increase (1.1-fold) in caspase 3 activity relative to control untreated cells was recorded. At both time points, the levels of caspase 3 activity in cells treated with Pi infusion alone was very similar to that measured in untreated cells. FIG. 3C presents the results of 20 hr exposure to H₂O₂ which demonstrates that the activity of caspase 3 is significantly elevated by H₂O₂ (p<0.001), and the induced activity is significantly (p<0.01) inhibited (49% inhibition of the induced activity) by Pi infusion.

### Example 4

Pi Infusion Reduces H₂O₂-Induced Increases in Intracellular Reactive Oxygen Species (ROS) Levels in Neuronal Cells

To assess the possibility that Pi infusion protects neuronal cells from H₂O₂-induced cell death by inhibiting H₂O₂-induced elevation of ROS levels, changes in intracellular levels of ROS were measured with the ROS indicator DCF-DA. Cells were labeled with (2′7′-dichlorofluorescein diacetate) DCF-DA and washed before being treated with increasing concentrations of Pi infusion for 2 h. Then, hydrogen peroxide (200 μM) was added to the medium and fluorescence levels were measured 1 h and 18 h thereafter.

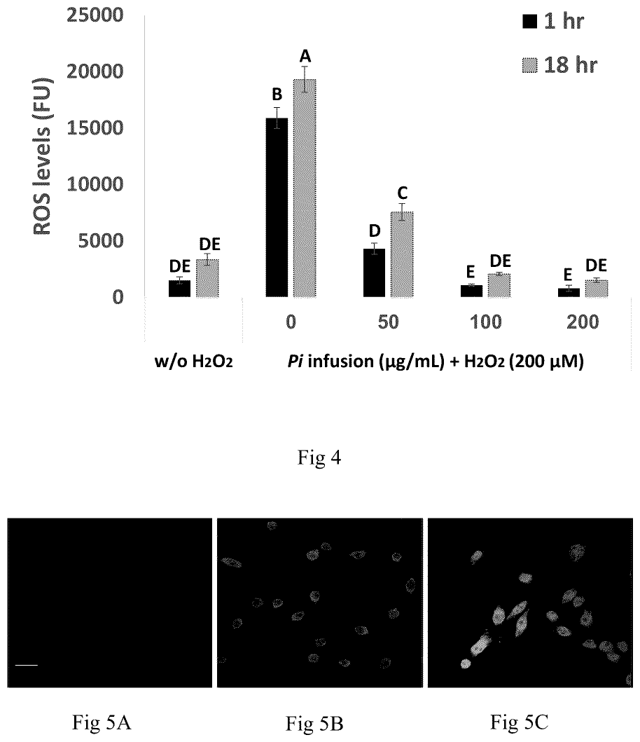

Treatment of SH-SY5Y cells with H₂O₂ (200 μM) resulted in a significant 10-fold and 6-fold elevation in intracellular ROS levels after 1 h and 18 h of treatment, respectively (FIG. 4), suggesting that elevation in ROS levels is an early event that persists for at least 18 h. When pretreated with various concentrations of Pi infusion 2 h before H₂O₂ application, the increase in H₂O₂-induced ROS levels was entirely abrogated (at 100 and 200 μg/mL), both after 1 h and 18 h of treatment with H₂O₂.

### Example 5

### Pi Infusion Upregulates Cellular Levels of Nrf2 and Induces its Translocation to the Nucleus

In the following example the effects of Pi infusion on the levels and localization of the Nrf2 transcription factor were examined. Under physiological conditions, most of the Nrf2 protein is in the cytoplasm. However, following appropriate signals, Nrf2 molecules translocate to the nucleus, where they activate the Nrf2-ARE pathway. Both immunostaining and biochemical assays were used to test whether Pi infusion activates the Nrf2 pathway.

SH-SY5Y cells were treated by Pi infusion (200 μg/mL) for 1 h or 2 h and then subjected to immunostaining for Nrf2. Nuclei were stained with DAPI. Two independent experiments were performed. Each experiment was conducted in duplicates.

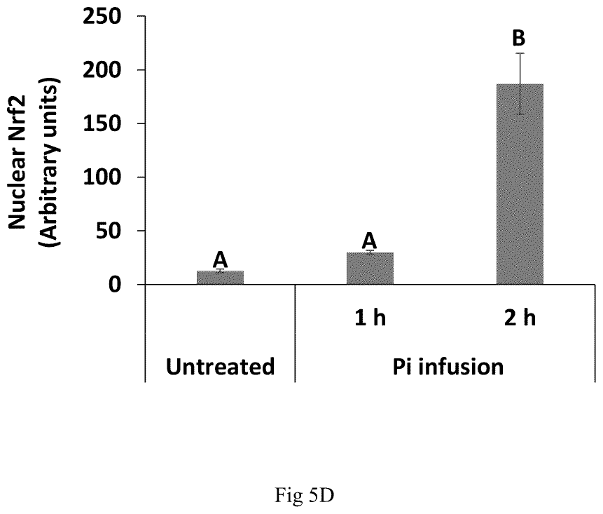

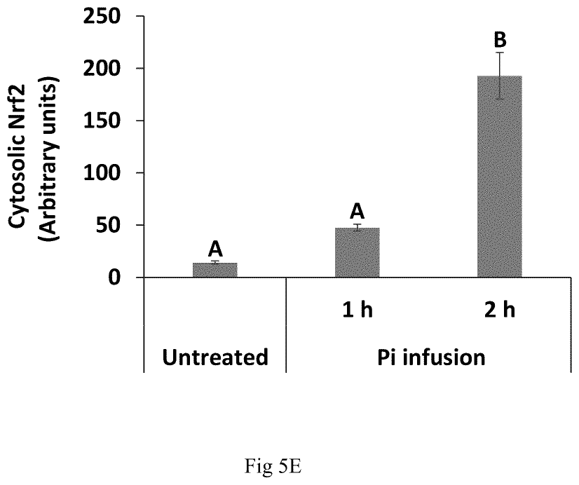

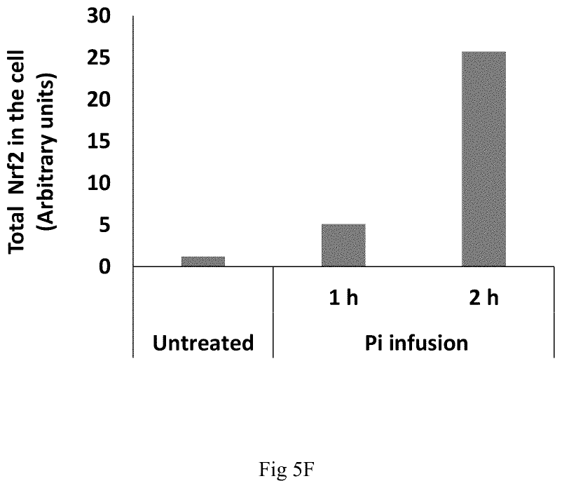

Immunostaining with anti-Nrf2 and DAPI demonstrated that in control untreated cells, Nrf2 levels were relatively very low (FIGS. 5A-5C). Following treatment with Pi infusion, cellular levels of Nrf2 were upregulated over time and underwent translocation from the cytoplasm to the nucleus. To quantify the effect of Pi infusion, the fluorescence intensities of Nrf2 in the nucleus and cytoplasm were normalized relative to the DAPI intensity. Normalized Nrf2 fluorescence of Pi-treated (2 h of treatment) as compared to untreated cells were 14-, and 15-fold higher in the cytoplasm, and nucleus, respectively (FIGS. 5D-5F).

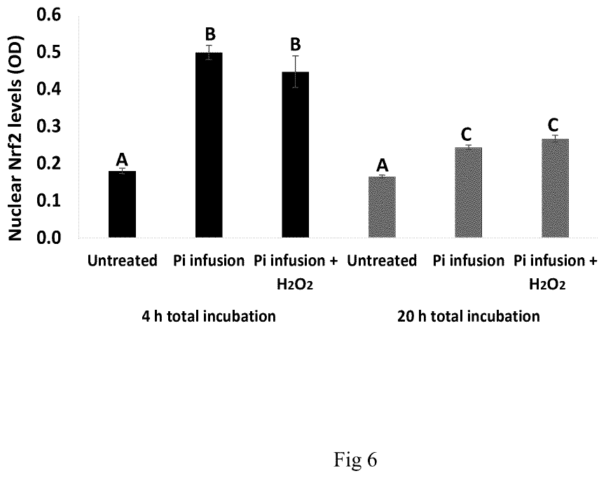

To validate these results, protein levels of nuclear Nrf2 were assessed using a biochemical kit 4 h and 20 h following exposure to Pi infusion. SH-SY5Y cells were pretreated with Pi infusion (200 μg/mL) for 2 h or 18 h, and then incubated for an additional 2 h with H₂O₂ (200 μM). Nrf2 levels in the nucleus were then determined. FIG. 6 shows that 4 h of treatment with Pi infusion increased nuclear Nrf2 levels 2.5-fold, relative to those of the control untreated cells. As expected, nuclear Nrf2 levels decreased with time, with Nrf2 levels after 4 h of Pi infusion treatment significantly higher than Nrf2 levels after 20 h of Pi infusion treatment. Next, the cells were preincubated with Pi infusion for 2 h and then treated with H₂O₂ for 2 h or 18 h. H₂O₂ had no significant effect on the Nrf2 levels that were induced by Pi infusion (FIG. 6).

### Example 6

### Pi Infusion Upregulates the Phosphorylation of the Transcription Factor Cyclic AMP Response Element-Binding Protein (CREB)

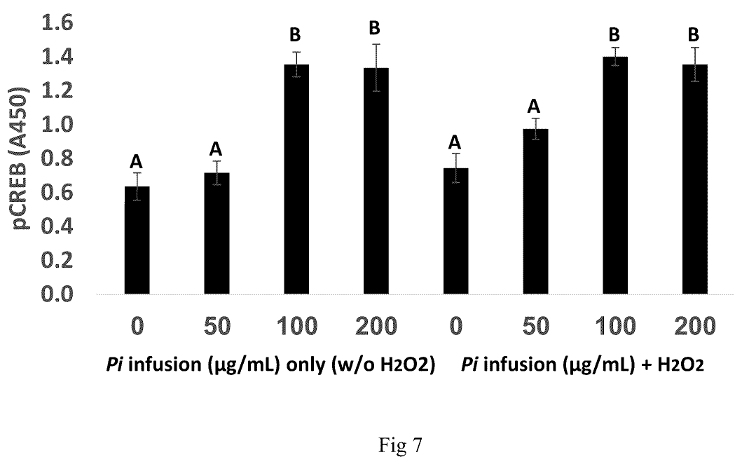

To gain more insight into the involvement of CREB signaling in the neuroprotective effect of Pi infusion, CREB phosphorylation was measured in neuronal cells treated with Pi infusion in the presence and absence of H₂O₂. Cells were pretreated for 2 h with increasing concentrations of Pi infusion. H₂O₂ (200 μM) was then added, and pCREB levels in cell lysates (25 μg) were determined 30 min later. Treatment of cells with Pi infusion for 2 h elicited a significant (p=0.001) increase in CREB phosphorylation, with a maximal 2-fold elevation at 100 μg/mL and 200 μg/mL infusion (FIG. 7) and did not change upon addition of H₂O₂. The optimal concentration for the induction of CREB phosphorylation was 100 μg/mL, which corresponded with the optimal concentration for cell protection from H₂O₂-induced cell death. It should be noted that treatment with Pi infusion and/or hydrogen peroxide did not induce an increase in the levels of total CREB.

### Example 7

### Consumption of Pi Infusion Prevents Neuronal Cell Death and Enhances Viability in the Hippocampus of Aging Mice

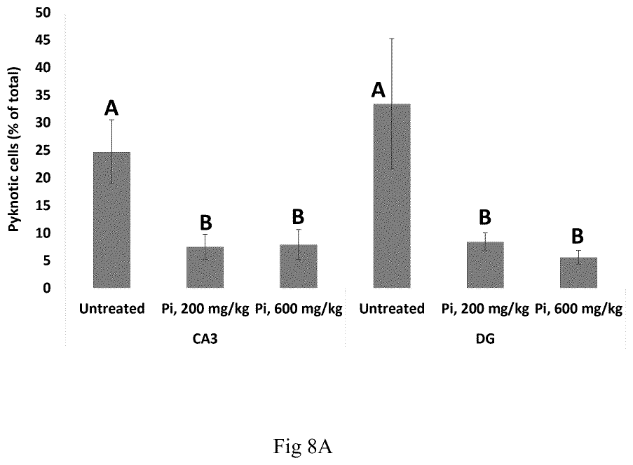

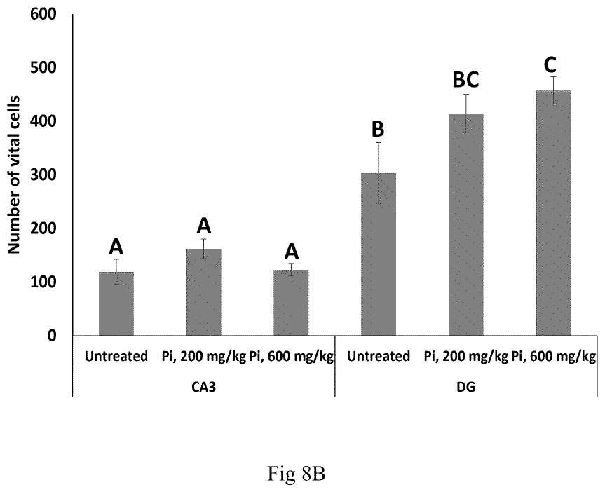

In the following example the potential neuroprotective effect of Pi infusion was investigated in vivo by testing its ability to prevent neuronal mortality in the hippocampus of aging mice. Brain sections of 19 months old mice treated daily for 18 months with Pi infusion, were stained with hematoxylin and eosin for morphometric analysis of the number of hippocampal neurons in the cornu Ammonis 3 (CA3) and dentate gyrus (DG) regions, as viewed through an objective magnification of ×20. The percentage of pyknotic neurons was calculated as the number of pyknotic neurons out of the total number of neuronal cells (vital+pyknotic). FIG. 8A demonstrates that after 18 months of daily treatment with Pi infusion-supplemented drinking water, mouse brains contained a significantly reduced percentage of pycnotic cells in the CA3 and DG (70% and 83% reduction, respectively). In the DG area, 600 mg/kg Pi infusion also significantly enhanced the number of vital neuronal cells by more than 50% (FIG. 8B).

### Example 8

### Pi Infusion Decreases Anxiety-Like Behavior in Adult Mice

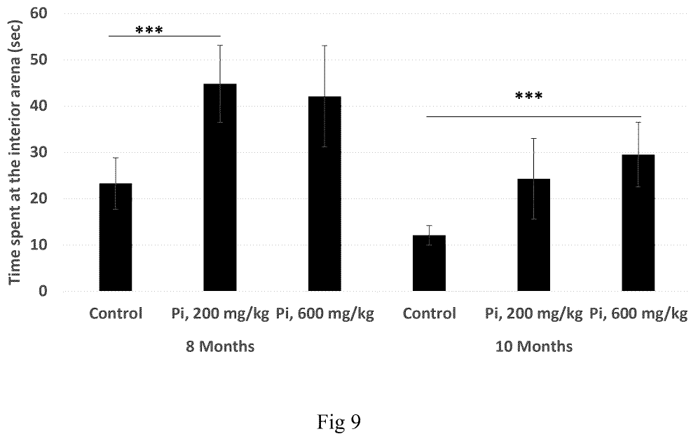

Open-Field Test Exploratory activity and anxiety-like behavior were measured using an open-field apparatus. Mice were subjected to the open-field test as described above. Distance traveled and time spent in the center (inner) zone by each animal was recorded for 10 min with the EthoVisionXT video-imaging system and compared between treatment groups. Time spent in the outer zones of the maze is indicative of anxiety-related behavior. Comparing time spent at the interior area during acquisition presented significant differences between treatment groups. As can be seen in FIG. 9, Mice treated with Pi infusion showed a significant elevation in time spent in the interior area as compared to the control untreated mice and hence exhibited less anxiety. 8 months old mice treated for 7 months with 200 mg/kg Pi infusion spent significantly more time in the interior area than the control mice. At the age of 10 months (9 months of consumption of Pi infusion), the time spent in the interior area was significantly higher in mice treated with 600 mg/kg Pi infusion compared to the control mice (p=0.02).

1-32. (canceled)

33. A pharmaceutical or nutritional composition for preventing, attenuating, or ameliorating the symptoms of neuronal cell death, or for reducing anxiety in a subject, wherein the composition comprises an infusion of the plant Pulicaria incisa (Pi) and one or more physiologically acceptable carriers.

34. The composition according to claim 33, wherein the subject has an age-related neuronal pathology.

35. The composition according to claim 34, wherein the age-related neuronal pathology comprises normal ageing of the brain.

36. The composition according to claim 35, wherein said normal ageing of the brain is characterized by an increase in neuronal disfunction and/or a reduction in neuronal viability.

37. The composition according to claim 33 wherein said subject is an elderly subject.

38. The composition according to claim 33 wherein said composition is in the form of a food article, a beverage, a food additive, a food supplement, or herbal drug.

39. The composition according to claim 33 wherein said composition is in the form of tablets, capsules, liquid syrups, nasal spray, nasal drops, soft gels, suppositories, patches, and enemas.

40. The composition according to claim 33 wherein said composition is administered at a concentration of between about 1 mg/kg and 100 mg/kg.

41. The composition according to claim 40 wherein said composition is provided orally at a concentration of between about 16 mg/kg and about 50 mg/kg.

42. The composition according to claim 33 wherein said composition is administered or consumed daily.

43. A method of preventing, attenuating, or ameliorating the symptoms of neuronal cell death, or reducing anxiety, wherein the method comprises administering to a subject in need thereof a therapeutically effective amount of an infusion of the plant Pulicaria incisa (Pi) or a composition comprising an infusion of the plant Pulicaria incisa (Pi).

44. The method according to claim 43, wherein the subject has an age-related neuronal pathology.

45. The method according to claim 44, wherein the age-related neuronal pathology comprises normal ageing of the brain.

46. The method according to claim 45, wherein said normal ageing of the brain is characterized by an increase in neuronal disfunction and/or a reduction in neuronal viability.

47. The method according to claim 43 wherein said subject is an elderly subject.

48. The method according to claim 43 wherein said composition is in the form of a food article, a beverage, a food additive, a food supplement, or herbal drug.

49. The method according to claim 43 wherein said composition is in the form of tablets, capsules, liquid syrups, nasal spray, nasal drops, soft gels, suppositories, patches, and enemas.

50. The method according to claim 43 wherein said composition is administered at a concentration of between about 1 mg/kg and 100 mg/kg.

51. The method according to claim 50 wherein said composition is provided orally at a concentration of between about 16 mg/kg and about 50 mg/kg.

52. The method according to claim 43 wherein said composition is administered or consumed daily.
